(19) United States
(12) Patent Application Publication
(10) Pub. No.: US 2016/0051734 A1
(43) Pub. Date: Feb. 25, 2016

(54) INWARDLY HYDROPHILICALLY COATED MEDICINAL-TECHNICAL DEVICE

(71) Applicant: ABAG AKTIENMARKT BETEILIGUNGS AG, Köln (DE)

(72) Inventor: Martin GÖRNE, Hamburg (DE)

(73) Assignee: ABAG AKTIENMARKT BETEILIGUNGS AG, Koeln (DE)

(21) Appl. No.: 14/784,625

(22) PCT Filed: Feb. 3, 2014
(86) PCT No.: PCT/EP2014/000277

§ 371 Date: Oct. 15, 2015

(30) Foreign Application Priority Data
Apr. 15, 2013 (EP) 13001970.6

(51) Int. Cl. A61L 31/10; C23C 16/02; C23C 16/50

(52) U.S. Cl. CPC A61L 31/10; C23C 16/50; C23C 16/0272; A61L 2420/02

(57) ABSTRACT

A method for coating a hollow body on its inner surface comprises pre-coating the inner surface with a reactive gas plasma (PECVD), and follow-up-coating the pre-coated surface with a reactive gas component without plasma action (CVD). The resulting hollow body is used for temporarily accommodating bodily liquids, in particular blood, or cell suspensions, due to its hydrophilizing PECVD/CVD-coating on its inner surface.

The present invention relates to an inwardly hydrophilically coated medicinal-technical device for temporally accommodating therein a bodily liquid, in particular blood, or a cell suspension. In particular, the device may be a bottle, a bag, a tube, or a pipe, but may also be an actuatable part of an apparatus for applying an external action, such as pressure, heat or radiation, on the bodily liquid or blood or cell suspension, respectively.

Devices for temporally accommodating blood are known, some of which are for flowing through and some of which are for temporal storage. As an example, the blood tube disclosed in the German laid-open patent application DE 10 2011 108 787 A1 may be mentioned, the content of which is herein disclosed by reference insofar relating to the die arrangement of a blood guiding system.

A general phenomenon of such devices is the increase of the tendency of the blood, or certain protein components in the bodily liquid, to coagulate due to the contact with the device. This effect in most applications is undesirable, and is counteracted, if necessary, by the administration of agents reducing the coagulation tendency. On the other hand, such agents have, as such, undesirable side effects; be it that blood preservations are used during a surgical operation, and the surgeon desires speedy stoppage of bleeding; or be it that a bodily liquid is desired to be returned or further processed as purely and uncontaminated as possible. Similar issues are encountered with cell suspensions intended for being administered to a human being or a vertebrate animal (or mammal).

It is known from the patent application US 2012/0123345 A1 to coat inner surfaces of vessels by inserting an electrode in den hollow space within the vessel, and igniting a plasma in the presence of a reactive gas. The (hollow) electrode simultaneously serves as a gas supply, while the (concave) counter electrode is arranged surrounding the vessel. With respect to the arrangement of the apparatus components, this patent application is likewise incorporated herein by reference. The express purpose of this known method is to provide water-repellent surfaces. The inwardly coated vessels obtained in this manner are, however, in practice not yet satisfactory for temporary accommodation of bodily liquids, blood, or cell suspensions, respectively.

The present invention aims at diminishing, reducing or avoiding the disadvantages of the known devices and methods. To this end, the invention, under a first aspect, provides an inwardly coated hollow body for temporarily accommodating a bodily liquid, in particular blood, or cell suspensions, the hollow body having a hydrophilizing PECVD/CVD-coating on its inner surface.

Under a further aspect, the invention provides a process for coating a hollow body at its inner surface, the method comprising:
pre-coating the inner surface using a reactive gas plasma (PECVD); and
follow-up-coating the pre-coated surface with a reactive gas component without plasma action (CVD).

Under a still further aspect, the invention provides a use of acrylic acid or acrylic acid anhydride for hydrophilizing surfaces of a body, the use comprising:
pre-coating the surfaces using a high frequency plasma generated from a gas mixture composed of an inert gas and a first gas formed of biocompatible, polymerizable, carboxy group-containing monomers, and then
follow-up coating the pre-coated surfaces of the body, wherein the pre-coating takes less than 10 minutes or even less than 1 minute, and the follow-up coating is made using a second gas substantially consisting of the acrylic acid or acrylic acid anhydride monomers.

Under the first aspect, a hollow body according to the invention has, at its inner surface, a hydrophilizing coating. Thereby, the tendency of the bodily liquid, blood, or cell suspension flown therethrough or accommodated therein is increased less than hitherto possible. In particular, a water contact angle, which designates the degree of hydrophily of a surface, may be set to less than 2° or even less than 0.5°. In embodiments, the surface material of the hollow body is a synthetic resin, and the hydrophilizing layer is made up of acrylic acid units. The hollow body may have exactly one opening, and may be a bottle or a bag; but it may also have two openings, through which, in use, the blood, bodily liquid, or cell suspension, respectively, is flown, and may be a pipe or a tube; or it may be an actuatable part of an apparatus applying, in operation, some external action, such as pressure, heat, or radiation, on the bodily liquid, blood, or cell suspension.

Under the second aspect, a method according to the invention may include an activating step, and includes a pre-treating step and a follow-up-treating step. Herein, in the activating step (if included), substantially only inert gas is present; in the pre-treating step, both inert gas as also a reactive gas is present; and in the terminal follow-up-treating step, substantially only the reactive gas is present, and there is no plasma action. In embodiments, the pre-treating takes less than 10 minutes, or less than 1 minute; the surface material of the hollow body may be made of a synthetic resin; and the coating material may be acrylic acid or its anhydride.

Without wishing to be bound by theory, it is assumed that the particularly short duration of the pre-treating step according to present invention allows for activated sites on the surface not to be immediately de-activated by the plasma, so that the two-step PECVD/CVD-coating particularly efficiently results in a hydrophilic surface.

Further advantages and features of the present invention will become apparent from the following detailed description of embodiments in conjunction with the drawings. The invention is not limited to the described embodiments, but by the scope of the appended claims. In particular, individual feature may be realized in embodiments according to the invention in a different number or and combination as in the examples described below. In the subsequent explanation of embodiments of the invention, reference is made to the accompanying drawings, which show:

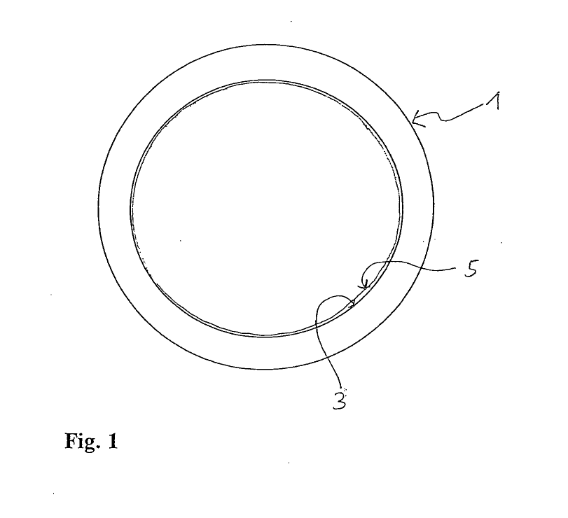

FIG. 1 a schematic cross-section through a hollow body according to the invention, and

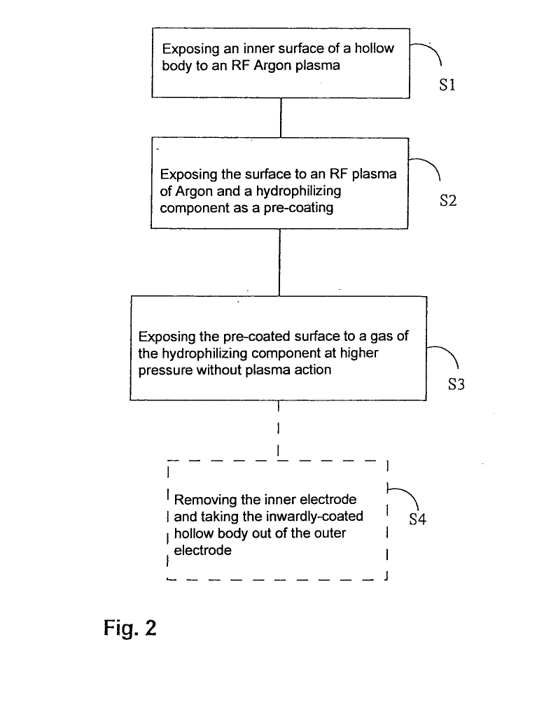

FIG. 2 a schematic flow diagram of a process according to the invention.

FIG. 1 depicts a generally oval, in the embodiment shown: substantially circular cross section of the hollow body 1. Although the wall is shown as being unitary and seamless, it may in actual fact be multi-layered and/or may have one or more seams. The inner surface layer 3 is formed of a synthetic resin, which term shall designate cross-linked or non-cross-linked polymeric materials, the latter in particular in conjunction with two-(or more)layer walls, in which the mechanical stability is provided mainly by the outer layer or layers. The water contact angle of the inner surface layer is 10° or more, in particular 30° or more, and the surface layer will be termed “little hydrophilic” in this context. Through the combined PECVD/CVD-coating process with ultra-short pre-coating, described in detail below, a very thin adhesion layer is formed on the inner surface and on top of that, a thicker function layer 5, both made of carboxy group-containing units, e. g. acrylic acid units. These layers are stably bound to the inner surface layer and are surprisingly extremely hydrophilic, notably more hydrophilic than those obtained in an otherwise similar manner, but with a longer (>1 minute, e.g. 10 minutes or more) pre-coating phase. It is thought that a longer pre-coating lets active sites, once generated, react further or with one another, in a manner detrimental to the hydrophilicity so obtainable. At the same time, a certain action time of the plasma is required for achieving a stable coating, such as at least 2 seconds.

The flow diagram scheme of FIG. 2 illustrates the important steps of a process for hydrophilizing inner surfaces by coating with poly(acrylic acid): After positioning a hollow electrode in the vessel or one-sidedly closed pipe or tube, same is evacuated by means of pumps, preferably to a pressure of maximally 10⁻⁴ mbar (10 mPa). After reaching the desired vacuum pressure, the interior of the vessel is purged with an inert gas, preferably Argon, while continuously pumping, wherein the inert gas supply is adjusted to the pumping speed so that a constant gas pressure is maintained in the interior of the vessel. The inert gas is supplied from an inert gas reservoir. In preferred embodiments, the Argon pressure so adjusted is about 25-150 mTorr (3.3-20 Pa). After reaching a stable inert gas pressure in the interior of the vessel, the plasma generator, for example a high frequency generator, is switched on for generating an inert gas plasma. The plasma cleans the inner surfaces by removing substances adsorbed thereto, and furthermore results in an activation of the inner surfaces by the formation of ions and free radicals, which enhance the subsequent graft polymerizing step. In some other embodiments, this pre-treatment step can be dispensed with.

The cleaning and activating effect in this first step Si may be influenced via the frequency of the gas plasmas, the power fed into the plasma, the exposition time to the plasma and the kind of the inert gas used for the plasma. As the inert gas, in the process proposed here Argon is preferred, because it allows for an activation of the inner surfaces without generating new, undesired compounds. Naturally, instead other inert gases such as N₂ may be used. In an exemplary embodiment of the process, the exposition time to the Argon plasma is 20 to 120 seconds. After the exposition, the process is continued with the first coating step S2. The plasma generator may be switched off temporarily if desired.

Deviating from the above, the plasma may be generated on the basis of a mixture of the inert gas and a reactive component used in a subsequent pre-coating step, instead of on the basis of pure Argon. The partial pressure of the reactive component in the gas mixture should in this case be less than one tenth of the partial pressure of the inert gas.

On transitioning from step S1 to step S2 of the process, the inert gas supply into the interior of the vessel is preferably maintained and desirably adjusted so that it assumes a value suitable for carrying out step S2. For generating the gas mixture, a first coating material gas formed of carboxy group-containing monomers in the gas phase is admixed to the inert gas. The carboxy group-containing monomers may preferably be acrylic acid or a precursor of acrylic acid, such as e.g. acrylic acid anhydride. The partial pressure p_{esG} of the first coating material gas in embodiments is at least one fourth of and maximally twice the partial pressure pₘ of the inert gas. It is particularly preferred that the partial pressure ratio p_{eSG}-p_{IG} is selected from a range of 1:1 to 1:0.5. For example, the partial pressure of Argon in embodiments of the process is 30 mTorr (ca. 400 mPa) at a total pressure of the gas mixture of 45 mTorr (ca. 600 mPa), resulting in a value of 2:1 for the ratio of the Argon partial pressure pₐᵣ to the partial pressure of the first coating material gas (reactive component partial pressure) p_{eSG}. While adjusting the desired reactive gas mixture the plasma generator may temporarily be switched off.

As the reactive component for forming the first coating material gas, preferably (meth)acrylic acid anhydride is used, which is evaporated in a separate container and is then guided to the interior of the vessel. The partial pressure of the coating material gas is adjusted via its supply, controlled by means of valves. Naturally, instead of (meth) acrylic acid anhydride (meth)acrylic acid may be used. (Meth)acrylic acid or (meth) acrylic acid anhydride, respectively, are provided in reservoir containers in liquid form, for example in an amount of 150 mL. In order to avoid or inhibit polymerisation of the acrylic acid or its precursor materials, respectively, same may be doted with Cu(I)-chloride. Furthermore, the reactive component containers are de-aerated after filling until no bubbles form in the reactive component liquid any longer. The vapour pressure of the reactive components usually is sufficient at normal room temperatures of 22 to 25° C. for generating the first coating material gas.

After the desired gas mixture and gas mixture pressure have been formed, which typically takes 1 to 2 minutes, the actual coating process is initiated by starting the plasma generator, whereby excited acrylic acid monomers generated in the plasma attach to the activated inner surface and, in the subsequent process, form a poly(acrylic acid) layer. This plasma-enhanced pre-coating phase (PECVD) is maintained only for a short time in order to form a very thin adhesion layer. The pre-coating phase may take less than 60, or between 2 and 20 seconds, resulting in a particularly low contact angle for water such as below 2° or even below 0.5°. The gas supplies are preferably not varied during the plasma coating. In a first variant of the process, the pre-coating process is terminated by switching off the plasma generator.

Subsequent to the described first variant of the pre-coating step S2, a first variant of the follow-up-coating step S3 follows, in which after switching off the plasma generator initially the inert gas supply is interrupted and the pre-coated inner surface is exposed to, if possible, the full vapour pressure of the reactive component formed of water-free acrylic acid. This vapour pressure should not be below ca. 5 Torr (670 Pa). Slightly cooling or warming the reactive component in the reservoir container may be suitable for adjusting the pressure. Supplying the reactive component into the interior of the vessel at full vapour pressure provides reactive gas in large amounts, which gas reacts with the reactive centres present at the pre-coated surface and forms a relatively thick poly(acrylic acid) layer (PAA-layer) which may be crystalline.

In a second variant of the process, the plasma generator is not switched off at the end of the pre-coating step S2 and is therefore still in operation at the transition to the second variant of the follow-up-coating step S3. In this variant, the Argon supply is stopped almost or entirely, and the supply of the reactive gas, e. g. of the acrylic acid, is reduced to such an extent that, while maintaining high-frequency generation and continuously evacuating the interior of the vessel, a pressure equilibrium in the range of less than 0.3 mTorr (ca. 40 mPa) is adjusted. In an exemplary embodiment, the pressure is set to a pressure of less than 0.1 mTorr (ca. 13 mPa). Thereby, the plasma extinguishes due to the reduction in pressure. Otherwise, the second variant is similar to the first variant.

In both variants, the follow-up-coating phase is maintained for 1 to 45, or for 5 to 15 minutes.

After termination of the process in step S4, the inwardly coated vessels can be taken out of the apparatus along with the hollow electrode, and the inward coating may be inspected for quality evaluation, if desired.

The process described above allows for a long-term-stable hydrophilization of polymeric inner surface of hollow bodies or vessels, respectively, having a low tendency of coagulation of the bodily liquids or cell suspensions flown therethrough, or temporarily stored therein, or acted upon externally while in the hollow body.

The skilled person will realize that numerous modifications and augmentations to the above described embodiments are possible without leaving the scope of protection of the appended claims.

For example, while the above description relates to inner surfaces of hollow bodies, the process may likewise be applied to bodies of any shape, such as convex bodies, parts or components e.g. intended for use inside a hollow body. As another example, while the above description advocates an initial pre-treatment step, such step may be dispensed with if the surfaces are already sufficiently clean. In such a case, the initial step is the pre-coating step, and there is no preceding step in which the amount of reactive gas or its partial pressure is less than one tenth of the inert gas (or, respectively, its partial pressure), or in which the reactive gas is even absent.

1. A hollow body, comprising a hydrophilic two-step PECVD/CVD-coating on its inner surface.

2-3. (canceled)

4. The hollow body according to claim 1, having a polymeric coating comprising acrylic acid monomers.

5. The hollow body according to claim 1, wherein a surface material of the hollow body is a synthetic resin.

6. The hollow body according to claim 1, having exactly one opening.

7. The hollow body according to claim 6, wherein the hollow body is a bag or a bottle.

8. The hollow body according to claim 1, having two openings, between which a bodily liquid or cell suspension, respectively, flows.

10. The hollow body according to claim 8, wherein the hollow body is an actuatable part of an apparatus adapted for applying an external action on the bodily liquid or the cell suspension.

11. A method for hydrophilically coating a hollow body for temporarily accommodating therein a bodily liquid at its inner surface, the method comprising:
pre-coating the inner surface by plasma-enhanced chemical vapor deposition (PECVD) with a reactive gas plasma; and then
follow-up-coating the pre-coated surface with a reactive gas component by chemical vapor deposition (CVD) without plasma action.

12. The method according to claim 11, wherein the pre-coating has a duration of less than 10 minutes or less than 1 minute.

13. The method according to claim 11, wherein the reactive gas component is acrylic acid or the anhydride thereof.

14. The method according to claim 11, wherein the surface material of the hollow body is a synthetic resin.

15. The method according to claim 11, wherein the pre-coating step is preceded by activating the inner surface with an inert gas plasma.

16. (canceled)

17. A method for hydrophilizing inner surfaces of a hollow body for temporarily accommodating therein a bodily liquid, the method comprising:
pre-coating the inner surfaces of the hollow body with a high frequency plasma generated from a gas mixture comprising an inert gas and a first gas formed of biocompatible, polymerizable, carboxy group-containing monomers; and then
follow-up coating the pre-coated surfaces of the hollow body,
wherein the pre-coating is performed for less than 10 minutes, and the follow-up coating is performed with a second gas consisting essentially of acrylic acid or acrylic anhydride monomers without plasma action.

18. The method of claim 17, wherein there is no preceding plasma-activation step in the absence of the first gas or with less than 10% of the gas formed of the monomers.

19. (canceled)

20. The method of claim 17, further comprising temporarily accommodating in the hollow body a bodily liquid or a cell suspension.

21. The method of claim 20, wherein the temporarily accommodating comprises filling the bodily liquid or cell suspension, respectively, into the hollow body, storing the bodily liquid or cell suspension, and later discharging the bodily liquid or cell suspension from the hollow body.

22. The method of claim 20, wherein the temporarily accommodating comprises steadily flowing the bodily liquid or cell suspension, respectively, through the hollow body.

23. The method of claim 20, comprising applying an external action selected from the group consisting of pressure, heat, and radiation on the bodily liquid or the cell suspension, respectively.

24. The method of claim 21, wherein the discharging the bodily liquid or cell suspension, respectively, from the hollow body comprises discharging the bodily liquid or cell suspension in an opposite direction to a direction of the filling.
